(19) United States
(12) Patent Application Publication
(10) Pub. No.: US 2021/0121124 A1
(43) Pub. Date: Apr. 29, 2021

(54) CLASSIFICATION MACHINE OF SPEECH/LINGUAL PATHOLOGIES

(71) Applicant: Ninispeech Ltd., Haifa (IL)

(72) Inventors: Itamar SHENHAR, Haifa (IL); Yoav MEDAN, Haifa (IL)

(21) Appl. No.: 17/046,774

(22) PCT Filed: Apr. 17, 2019
(86) PCT No.: PCT/IL2019/050435

§ 371 Date: Oct. 10, 2020

Related U.S. Application Data
(60) Provisional application No. 62/662,519, filed on Apr. 25, 2018.

(51) Int. Cl. A61B 5/00; A61B 5/16; G06N 3/08; G06N 20/10; G10L 25/66; G10L 25/63; G10L 25/30

(52) U.S. Cl. CPC A61B 5/4803; A61B 5/7267; A61B 5/486; A61B 5/165; G10L 25/30; G06N 20/10; G10L 25/66; G10L 25/63; G06N 3/08

(57) ABSTRACT

There is provided herein a method for treating/diagnosing a speech/language related pathology, the method comprising: introducing a speech sample provided by a user to a speech/language machine learning (ML) classifier, wherein the ML classifier is trained with non-pathological/normal speech, applying novelty detection algorithms to compute a similarity measure, and based at least on the similarity measure, computing an output signal indicative of a speech/lingual quality of the user.

### FIELD OF THE INVENTION

Embodiments of the disclosure relate to speech/language pathologies.

### BACKGROUND

Traditionally, classification of speech pathologies for diagnosis and assessment of therapy progress are done subjectively by a trained human professional. More recently, computers have shown to be reliably capable of understanding human speech, using new approaches that rely on vast amount of tagged speech data (the text encoding and time alignment are known) and processing power. Such classification machines are various variants of what is called Deep Neural Networks (DNNs). Still, they fall short in classifying and understanding pathological speech and thus, are unable to diagnose and assess the quality of such speech.

There is a need in the art for improved and efficient methods and systems for diagnosing and treating speech/language related pathologies.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

Initial attempts to bridge the gap between classification of normal speech and understanding pathological speech were based on analyzing the speech and applying a set of rules for detecting pathological events such as in stuttering. However, to improve the robustness of such classification machine and broaden its scope to other speech pathologies, such as, but not limited to, articulation, one would need large sets of high quality tagged pathological speech data, which do not currently exist and would cost a lot of resources to acquire.

There still exists a large gap of insufficient data for training a deep neural network based classification machines in the field of speech/language pathologies.

There are thus provided herein, according to some embodiments, a method and system that eliminate the need for a large amount of tagged speech training (pathological speech samples). According to some embodiments, training of a Neural Network (NN) classifier, such as RNN auto-encoder with bidirectional LSTM units, is performed using vast amounts of non-pathological/normal speech, with MFCC features concatenated with their first- and second-order derivatives as inputs. Then, according to further embodiments, the auto-encoder measures the degree of similarity of a given new speech sample to normal speech. Thus, feeding a pathological speech sample will cause a deterioration in the similarity measure, since such samples have never been introduced (or very rarely introduced) during a training phase and constitute an outlier.

According to some embodiments, such a classifier may be language-agnostic since it is not necessarily aimed at understanding the speech but rather its prosody and/or basic sound units.

According to additional embodiments, a secondary classifier may be added. The secondary classifier is utilized for sub-classifying the speech that has been tagged as pathological, into a sub-class category such as stuttering, articulatory, Aphasia, Parkinson, etc.

According to some embodiments, such a secondary classifier can be, implemented using various known Machine Learning (ML) techniques (DNN, RNN, SVM, KNN, etc.).

There is thus provided herein, according to some embodiments, a method for treating/diagnosing a speech/language related pathology, the method comprising: introducing a speech sample provided by a user to a speech/language machine learning (ML) classifier, wherein the ML classifier is trained with non-pathological/normal speech; applying novelty detection algorithms to compute a similarity measure; and based at least on the similarity measure, computing an output signal indicative of a speech/lingual quality of the user.

There is thus provided herein, according to some embodiments, a computer implemented method for treating/diagnosing a speech/language related pathology, the method comprising: introducing a speech sample provided by a user to a speech/language machine learning (ML) classifier, wherein the ML classifier is trained with non-pathological/normal speech; applying novelty detection algorithms to compute a similarity measure; and based at least on the similarity measure, computing an output signal indicative of a speech/lingual quality of the user.

There is further provided herein, according to some embodiments, an electronic device comprising one or more processors; and memory coupled to the one or more processors, the memory storing one or more programs configured to be executed by the one or more processors, the one or more programs including instructions for: introducing a speech sample provided by a user to a speech/language machine learning (ML) classifier, wherein the ML classifier is trained with non-pathological/normal speech; applying novelty detection algorithms to compute a similarity measure; and based at least on the similarity measure, computing an output signal indicative of a speech/lingual quality of the user.

There is further provided herein, according to some embodiments, a system for treating/diagnosing a speech/language related pathology, the system comprising: one or more processors configured to: introduce a speech sample provided by a user to a speech/language machine learning (ML) classifier, wherein the ML classifier is trained with non-pathological/normal speech; apply novelty detection algorithms to compute a similarity measure; and based at least on the similarity measure, compute an output signal indicative of a speech/lingual quality of the user; and a recorder configured to configured to record the speech sample provided by the user.

According to some embodiments, the ML classifier may apply deep neural network (DNN) support vector machine (SVM), (k-nearest neighbors) KNN algorithms or any combination thereof. According to some embodiments, the DNN algorithms may include recurrent neural networks (RNNs), convolutional deep neural networks (CNNs) or a combination thereof.

According to some embodiments, the method may further include tagging the speech sample as normal if the similarity measure is at or above a predetermined threshold and tagging the speech sample as abnormal if the similarity measure is below the predetermined threshold.

According to some embodiments, the step of computing a speech/lingual quality of the user may further include collecting a duration of abnormal speech intervals and/or a duration of normal speech intervals.

According to some embodiments, the method may further include applying ML algorithms for sub-classifying speech tagged as abnormal. The ML sub-classifying may apply deep neural network (DNN) support vector machine (SVM), (k-nearest neighbors) KNN algorithms or any combination thereof. The DNN algorithms may include recurrent neural networks (RNNs), convolutional deep neural networks (CNNs) or a combination thereof.

According to some embodiments, the output signal may further include one or more assigned speech/lingual quality scores.

According to some embodiments, the speech/lingual quality may include one or more speech qualities may include speech intelligibility, fluency, vocabulary, accent, emotion, pronunciation, jitter, shimmer, duration, intonation, tone, rhythm, and any combination thereof.

According to some embodiments, the speech/lingual quality may include one or more speech qualities may include one or more lingual qualities selected from a group consisting of: comprehension, pronunciation, planning and/or organization of correct grammar, pragmatic skills of communication, and any combination thereof.

According to some embodiments, the method may further include providing a feedback signal to the user and/or to a caregiver.

More details and features of the current invention and its embodiments may be found in the description and the attached drawings.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. It is intended that the embodiments and figures disclosed herein are to be considered illustrative rather than restrictive. The figures are listed below:

FIG. 1 schematically depicts a block diagram of a system for treating/diagnosing a speech/language related pathology, according to some embodiments; and

FIG. 2 schematically depicts a flowchart of a method for treating/diagnosing a speech/language related pathology, according to some embodiments.

### DETAILED DESCRIPTION

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

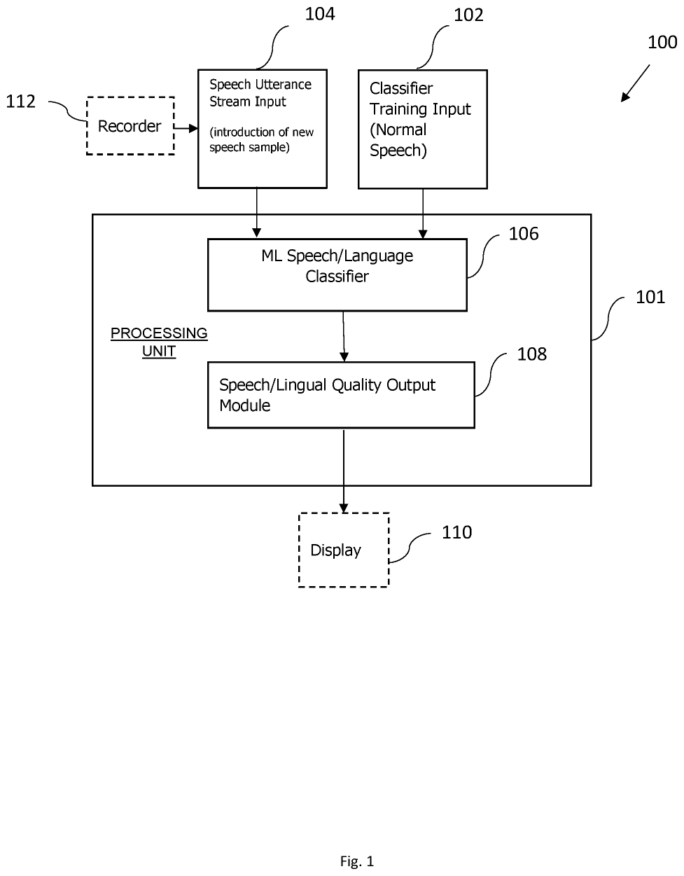

Reference is now made FIG. 1, which schematically depicts a block diagram of a system 100 for treating/diagnosing a speech/language related pathology, according to some embodiments.

System 100 includes a processing unit 101, which includes a speech/language classifier 106 and a speech/lingual quality output module 108. Speech/language classifier 106 is configured to be trained with non-pathological/normal speech, introduced thereto by classifier training input 102. After speech/language classifier 106 is trained with normal speech, a new speech sample is introduced to speech/language classifier 106 by “Speech Utterance Stream Input” 104. Speech/language classifier 106 applies novelty detection algorithms (e.g., RNN auto-encoder based algorithms) to the speech sample in order to identify novel patterns. If a novel pattern is detected, the speech is tagged as abnormal. If a novel pattern is not detected, the speech is tagged as normal. In other words, speech/language classifier 106 computes a similarity measure. The classifier outputs a degree of similarity to trained samples, for example in a scale of 0%-100%. The higher the value of the similarity measure, the higher the likelihood of similarity to trained samples (in other words, the new speech sample is tagged as normal), and vice versa, the lower the value of the similarity measure, the lower the likelihood that the new speech sample is tagged as normal (the system has not heard it before), i.e., the new speech sample is tagged as abnormal.

Small values indicate novelty (have not heard it before) while large values indicate high likelihood of similarity to trained samples.

The duration of all normal and abnormal intervals are separately collected and a speech/lingual quality of a user is computed by speech/lingual quality output module 108 and optionally displayed by display 110.

System 100 may further include a recorder 112 configured to record a speech sample of a user and to introduce it to speech/language classifier 106.

It is noted, according to some embodiments, that a speech/language classifier such as 106, is not trained by the speech utterance stream (i.e., the new, potentially abnormal, speech samples) introduced thereto. In other words, when a user's potentially abnormal speech sample is introduced to the speech/language classifier it does not train the system. This is to allow the classifier to keep identifying abnormal speech samples as novel.

However, after a speech sample is tagged as abnormal, sub-classifying machine learning algorithms may be applied and the system keeps training by every speech sample tagged as abnormal. Moreover, according to some embodiments, sample tagged as abnormal by a speech/language classifier such as 106, may now be re-tagged (corrected) as normal.

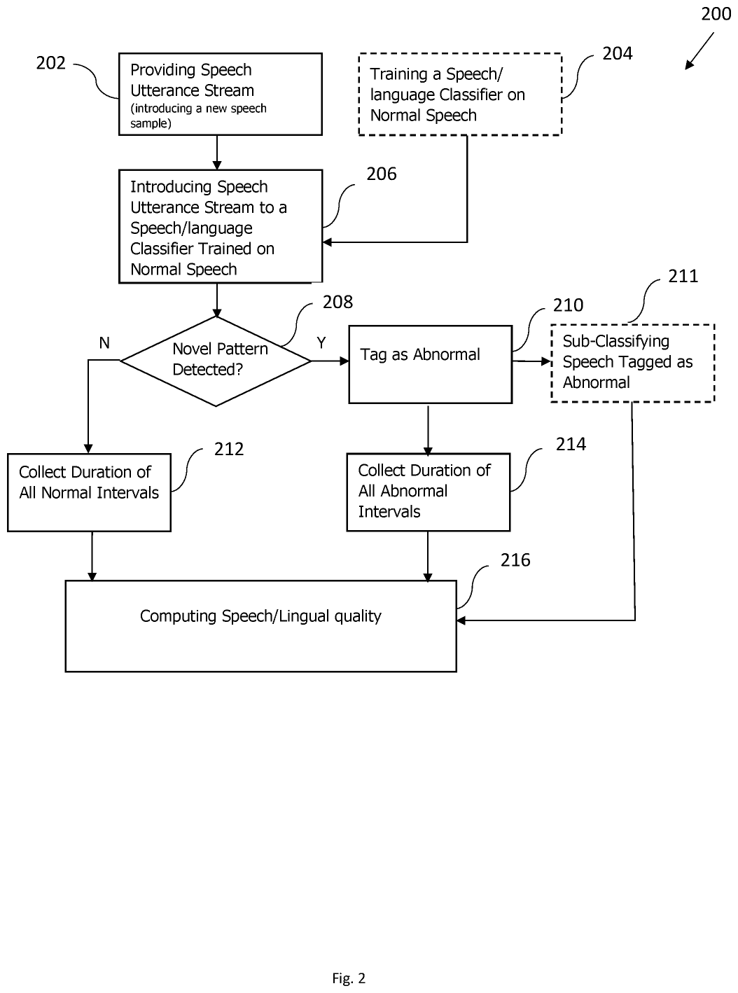

Reference is now made FIG. 2, which schematically depicts a flowchart 200 of a method for treating/diagnosing a speech/language related pathology, according to some embodiments. The method includes the following steps:

Step 202—Providing speech utterance stream obtained from a subject suspected of having a speech/language pathology, for example but not limited to, a subject suffering from speech/language behavioral, developmental, rehabilitation and/or degenerative conditions/diseases. Example of conditions/diseases may include aphasia, Parkinson, Alzheimer's, stuttering etc.

Step 206—speech utterance stream is introduced to a speech/language classifier which was previously trained on normal speech (Step 204).

Step 208—once the speech utterance stream was introduced to the speech/language classifier, the system applies novelty detection algorithms to the speech in order to identify novel patterns.

It is noted, according to some embodiments, that this speech/language classifier, is not trained by the speech utterance stream (i.e., the new, potentially abnormal, speech samples) introduced thereto. In other words, when a user's potentially abnormal speech sample is introduced to the speech/language classifier it does not train the system. This is to allow the classifier to keep identifying abnormal speech samples as novel.

If novel pattern is detected, the speech is tagged as abnormal (Step 110) and the duration of all abnormal intervals is collected (Step 114). If novel pattern is not detected, the speech is tagged as normal and the duration of all normal intervals is collected (Step 112). Based on the normal intervals duration and the abnormal intervals duration a speech/lingual quality is computed (Step 116) and optionally displayed.

Optionally, Step 211 may also be performed. Step 211 includes sub-classifying speech tagged as abnormal (in Step 210). In Step 211, i.e., after a speech sample is tagged as abnormal, sub-classifying machine learning algorithms may be applied and the system keeps training by every speech sample tagged as abnormal. Moreover, according to some embodiments, sample tagged as abnormal (e.g., in steps 208, 210), may now be re-tagged (corrected) as normal.

Sub-classifying the speech that has been tagged as pathological, into a sub-class category such as stuttering, articulatory pathology, Aphasia related speech/lingual pathology, Parkinson related speech/lingual pathology, etc.

According to some embodiments, such a secondary classifier can be implemented using various known ML techniques (such as but not limited to, DNN, SVM, KNN).

In other words, the system computes a similarity measure. The higher the value of the similarity measure, the higher the likelihood that the speech utterance stream is tagged as normal, and vice versa, the lower the value of the similarity measure, the lower the likelihood that the speech is tagged as normal, in other words, the speech is tagged as abnormal.

In the description and claims of the application, each of the words “comprise” “include” and “have”, and forms thereof, are not necessarily limited to members in a list with which the words may be associated.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What we claim is:

1. A method for treating/diagnosing a speech/language related pathology, the method comprising:
introducing a speech sample provided by a user to a speech/language machine learning (ML) classifier, wherein the ML classifier is trained with non-pathological/normal speech;
applying novelty detection algorithms to compute a similarity measure; and
based at least on the similarity measure, computing an output signal indicative of a speech/lingual quality of the user.

2. The method of claim 1, wherein the ML classifier applies deep neural network (DNN) support vector machine (SVM), (k-nearest neighbors) KNN algorithms or any combination thereof.

3. The method of claim 2, wherein the DNN algorithms comprise recurrent neural networks (RNNs), convolutional deep neural networks (CNNs) or a combination thereof.

4. The method of claim 1, further comprising tagging the speech sample as normal if the similarity measure is at or above a predetermined threshold and tagging the speech sample as abnormal if the similarity measure is below the predetermined threshold.

5. The method of claim 1, wherein the step of computing a speech/lingual quality of the user further comprising collecting a duration of abnormal speech intervals and/or a duration of normal speech intervals.

6. The method of claim 4, further comprising applying ML algorithms for sub-classifying speech tagged as abnormal.

7. The method of claim 6, wherein the ML sub-classifying applies deep neural network (DNN) support vector machine (SVM), (k-nearest neighbors) KNN algorithms or any combination thereof.

8. The method of claim 7, wherein the DNN algorithms comprise recurrent neural networks (RNNs), convolutional deep neural networks (CNNs) or a combination thereof.

9. The method of any one of claims 1-8, wherein the output signal further comprises one or more assigned speech/lingual quality scores.

10. The method of any one of claims 1-9, wherein the speech/lingual quality comprises one or more speech qualities selected from a group consisting of: speech intelligibility, fluency, vocabulary, accent, emotion, pronunciation, jitter, shimmer, duration, intonation, tone, rhythm, and any combination thereof.

11. The method of any one of claims 1-10, wherein the wherein the speech/lingual quality comprises one or more lingual qualities selected from a group consisting of: comprehension, pronunciation, planning and/or organization of correct grammar, pragmatic skills of communication, and any combination thereof.

12. The method of any one of claims 1-11, further comprising providing a feedback signal to the user and/or to a caregiver.

13. An electronic device comprising one or more processors; and memory coupled to the one or more processors, the memory storing one or more programs configured to be executed by the one or more processors, the one or more programs including instructions for:
introducing a speech sample provided by a user to a speech/language machine learning (ML) classifier, wherein the ML classifier is trained with non-pathological/normal speech;
applying novelty detection algorithms to compute a similarity measure; and
based at least on the similarity measure, computing an output signal indicative of a speech/lingual quality of the user.

14. A system for treating/diagnosing a speech/language related pathology, the system comprising:
one or more processors configured to:
introduce a speech sample provided by a user to a speech/language machine learning (ML) classifier, wherein the ML classifier is trained with non-pathological/normal speech;
apply novelty detection algorithms to compute a similarity measure; and
based at least on the similarity measure, compute an output signal indicative of a speech/lingual quality of the user; and
a recorder configured to configured to record the speech sample provided by the user.
